(19) United States
(12) Patent Application Publication
(10) Pub. No.: US 2019/0209872 A1
(43) Pub. Date: Jul. 11, 2019

(54) DETECTION OF TREATMENT FAILURE FOR MILD HYPERTHERMIA

(71) Applicant: KONINKLIJKE PHILIPS N.V., EINDHOVEN (NL)

(72) Inventors: Robert Michael STARUCH, DALLAS, TX (US); Shyam BHARAT, ARLINGTON, MA (US); Shriram SETHURAMAN, LEXINGTON, MA (US); Vijay PARTHASARATHY, LEXINGTON, MA (US); Ajay ANAND, ROCHESTER, NY (US); Ehsan DEHGHAN MARVAST, PALO ALTO, CA (US)

(21) Appl. No.: 16/326,258

(22) PCT Filed: Aug. 17, 2017
(86) PCT No.: PCT/EP2017/070887

§ 371 Date: Feb. 18, 2019

Related U.S. Application Data
(60) Provisional application No. 62/379,786, filed on Aug. 26, 2016.

(51) Int. Cl. A61N 7/02; A61B 5/01; A61B 8/08; A61B 5/00; A61B 5/055; G01R 33/48

(52) U.S. Cl. CPC A61N 7/02; A61B 5/015; A61B 8/485; A61N 2007/0052; A61B 5/055; G01R 33/4804; G01R 33/4814; A61B 5/0036

(57) ABSTRACT

A system (100) includes an imaging system (130), and a therapy control device (122). The imaging system (130) generates temperature maps (140) and strain maps (142) of localized tissues of a patient. The therapy control device (122) includes one or more computer processors configured to detect at least one failure mode (300, 302, 304, 400) of generated mild hyperthermia in the localized tissues of the patient according to at least one of the temperature maps, the strain maps, or a signal indicative of detected inertial cavitation. In some embodiments, the therapy control device either halts therapy or issues a warning.

### FIELD OF THE INVENTION

The following generally relates to mild hyperthermia treatments and more specifically to detection of treatment failures in mild hyperthermia treatments.

### BACKGROUND OF THE INVENTION

Mild hyperthermia, as a treatment mode, is a localized therapeutic heating of diseased, often cancerous, tissue in a patient to 40-43° C. for a predetermined time of at least 10 minutes, e.g. 10-60 minutes. Mild hyperthermia is used as an adjuvant therapy to a primary therapy, such as radiotherapy or chemotherapy. For example, heating sensitizes the localized tissue to the primary therapy when delivered within 2 hours before, during, or within 2 hours after the primary therapy.

Mild hyperthermia is different from hyperthermic ablative treatments, which destroy tissue. At higher temperatures heated tissues are destroyed and/or damaged. Using ultrasound to provide localized mild hyperthermia allows the primary therapy to specifically and precisely target diseased tissue separately from normal tissue within the localized tissue.

Ultrasound delivery of mild hyperthermia can be monitored with an imaging modality such as ultrasound or magnetic resonance. Both imaging modalities can provide temperature maps. Ultrasound typically can also provide strain maps. Magnetic resonance when configured with additional equipment, such as an external actuator, can also provide strain maps. Temperature maps can provide tissue temperatures of each spatial location according to a voxel in an image of the patient. Strain maps can provide elasticity measurements of each spatial location according to a voxel in an image of the patient. The temperature map is typically used to orient the therapeutic heating of one or more predetermined regions of the patient, which include the localized tissues to be treated.

The tissues of the body are not uniform, and the mechanisms of the body naturally work to maintain homeostasis. As a result, in the delivery of mild hyperthermic heating by ultrasound to tissues of the body, it may be difficult to maintain the temperature of all the tissues in the region in the range of 40-43° C. for a designated time, e.g. using a known temperature monitoring approach, such as surgically inserting thermocouples into the localized tissue. As a consequence, some of the tissue may undergo irreversible thermal damage.

### SUMMARY OF THE INVENTION

Aspects described herein address the above-referenced problems and others.

The following describes ultrasound generated mild hyperthermia in a patient with detection of treatment failure modes. The treatment failure modes include detection within a localized region at least one of increased tissue stiffness, temperature above a threshold, cavitation, and/or image monitoring signal loss. A detected treatment failure mode halts the ultrasound based mild hyperthermia. The detected treatment failure modes can include treatment failure mode warnings.

In one aspect, a system includes an imaging system, and a therapy control device. The imaging system generates temperature maps and strain maps of localized tissues of the patient. The therapy control device includes one or more computer processors configured to detect at least one treatment failure mode of generated mild hyperthermia in the localized tissues of the patient according to at least one of the temperature maps, the strain maps, or a signal indicative of detected inertial cavitation.

In another aspect, a method includes generating temperature maps and strain maps of localized tissues of a patient. At least one treatment failure mode is detected of generated mild hyperthermia in the localized tissues of the patient according to at least one of the temperature maps, the strain maps, or a signal indicative of detected inertial cavitation.

In another aspect, a system includes a high intensity focused ultrasound (HIFU) transducer, an imaging system, a passive cavitation detector, and a therapy control device. The HIFU transducer generates mild hyperthermia with high intensity focused ultrasound in localized tissues of a patient. The imaging system generates temperature maps and strain maps of the localized tissues of the patient. The passive cavitation detector detects inertial cavitation within the localized tissues of the patient and generates a signal indicative of the detected inertial cavitation. The therapy control device includes one or more computer processors configured to detect at least one treatment failure mode of the generated mild hyperthermia in the localized tissues of the patient according to at least one of the temperature maps, the strain maps, or the signal indicative of the detected inertial cavitation. In response to the at least one detected treatment failure mode, the therapy control device halts the generated mild hyperthermia by the HIFU transducer.

### BRIEF DESCRIPTION OF THE DRAWINGS

The invention may take form in various components and arrangements of components, and in various steps and arrangements of steps. The drawings are only for purposes of illustrating the preferred embodiments and are not to be construed as limiting the invention.

FIG. 1 schematically illustrates an embodiment of a system that includes ultrasound hyperthermia treatment with detection of treatment failure modes.

FIG. 2 illustrates an example system graphical user interface of the ultrasound hyperthermia treatment system with detection of treatment failure modes.

FIG. 3 flowcharts an embodiment of a method for detecting a treatment failure mode for mild hyperthermia.

FIG. 4 flowcharts an embodiment of a method for detecting a treatment failure mode for mild hyperthermia.

FIG. 5 flowcharts an embodiment of a method of ultrasound mild hyperthermia treatments with detected treatment failure modes.

### DETAILED DESCRIPTION OF EMBODIMENTS

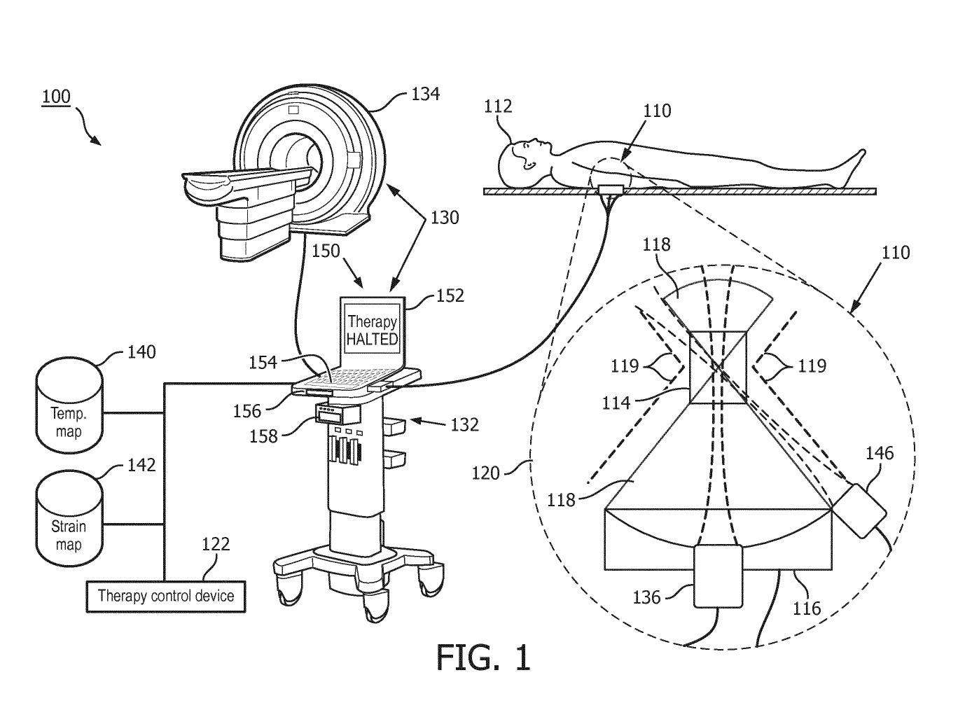

Initially referring to FIG. 1, an embodiment of a system 100 that includes ultrasound hyperthermia treatment with detection of treatment failure modes is schematically illustrated with an exploded view of a localized treatment region 110 of a patient 112. A HIFU transducer 116 is configured to focus ultrasound energy into a target region 114 and by proximity into warning regions 118, which are adjacent regions or regions between the target region 114 and the HIFU transducer 116 in a path of the focused ultrasound energy. In one embodiment the warning regions 118 are defined by the path of the focused ultrasound energy. In another embodiment, the warning regions 118 include a margin 119, such as an adjacent region to the region of the path of the focused ultrasound energy and the target region 114. In some embodiments, the warning regions 118 are shaped in an hour glass shape of an expected acoustic field that includes the target region 114, the regions defined by the path of the focused ultrasound energy and some additional adjacent regions to the target region 114 and the regions defined by the path of the focused ultrasound energy.

The HIFU transducer 116 is configured to heat and maintain the target region 114 within a range of temperatures intended to augment or localize the effects of a primary therapy, such as 40-45° C., 43-45° C. or 40-43° C. The localized treatment region 110 can include untreated regions 120 which are adjacent to the target region 114 and/or the warning regions 118. The delivery of the ultrasound energy is controlled by a therapy control device 122, which detects treatment failure modes. The therapy control device 122 halts therapy in the event that a treatment failure mode is detected. The therapy control device 122 can provide treatment failure mode warnings. In some embodiments, the therapy control device 122 adjusts parameters, controls and/or direction of the focused ultrasound energy by the HIFU transducer 116 based on the spatial location in the localized treatment region 110 according to the treatment failure mode warnings. In some embodiments, the healthcare practitioner adjusts the parameters, the controls, and/or the direction of the focused ultrasound energy based on the spatial location in the localized treatment region 110 according to the treatment failure mode warnings. In some embodiments the therapy control device 122 adjusts parameters, controls and/or direction of the focused ultrasound energy by the HIFU transducer 116 based on the spatial location in the localized treatment region 110 according to the treatment failure mode warnings, and the healthcare practitioner confirms, rejects, or modifies the adjustments by the therapy control device 122, or removes the patient 112 from treatment.

An imaging system 130, such as an ultrasound (US) imaging system 132 and/or a magnetic resonance (MR) imaging system 134, is used to generate volumetric imaging data of the localized treatment region 110. The imaging system 130 generated imaging data can include a signal indicating no current imaging data is available, e.g. loss of signal. In one embodiment, an ultrasound imaging probe 136 of the US imaging system 132 is used to generate the imaging data and the MR imaging system 134 can be omitted. In another embodiment, a whole body coil or local coil of the magnetic resonance imaging system 134 is used to generate the imaging data and the ultrasound imaging probe 136 can be omitted. In another embodiment, a combination of the US imaging system 132 and the MR imaging system 134 generate imaging data. The imaging system 130 is configured to generate from the imaging data, a temperature map 140 and a strain map 142 using US and/or MR techniques known in the art, such as “Apparatus and method for computing 3D ultrasound elasticity images” [U.S. Pat. Pub. 2008/0306384], “Devices, methods, and systems for measuring elastic properties of biological tissues” [WIPO Pub. WO/2011/153268], “Magnetic resonance elastography using multiple drivers” [U.S. Pat. 7307423], and the like. The temperature map 140 provides tissue temperatures for voxel locations in the localized treatment region 110. The strain map 142 provides tissue elasticity measures for each voxel location in the localized treatment region 110. The imaging system 130 can further provide an anatomical image of the localized treatment region 110.

A passive cavitation detector (PCD) 146 generates a signal indicative of cavitation in the localized treatment region 110. For example, the signal includes an indicator of backscatter from the HIFU ultrasound or an indicator of broadband emissions. In one embodiment the signal of the PCD 146 can include a cavitation map of the localized treatment region 110.

The therapy control device 122 detects treatment failure modes which include increased tissue stiffness, temperature above a threshold, cavitation, and/or image monitoring signal loss or corruption. In some instances, increased tissue stiffness, a temperature above a threshold, and/or a cavitation indicates tissue damage. In some instances an imaging signal loss or corruption indicates an unacceptable risk of tissue damage. The therapy control device 122 determines increased tissue stiffness from the strain map 142, which can include one or more strain maps 142 over time. The therapy control device 122 determines temperatures above a threshold from the temperature map 140, which can include one or more temperatures maps 142 over time. For example, the temperature maps are accurate to within ±1° C., and each voxel includes a temperature value that can change with refresh of the temperature map. The therapy control device 122 determines cavitation from the cavitation signal of the PCD 146. The maps can be refreshed in an interval between one and ten seconds. The maps can be refreshed in less than two to three seconds, such as from the MR imaging system 134, and can be refreshed approximately ten to twenty (e.g. 10-50+) times per second, such as from the US imaging system. The therapy control device 122 determines image monitoring signal loss and/or corruption from the generated imaging data of the imaging system 130.

The console 150 includes a display device 152, such as a computer display, projector, body worn display, and the like, and one or more input devices 154, such as a mouse, keyboard, microphone, touch or gesture interface, and the like. The console 150 with the therapy control device provides a user interface for a healthcare practitioner to interact with the system through the display device 152 and the input device 154. The user interface allows control and monitoring of delivery of the ultrasound energy and provides notice of detected treatment failure modes. The console 150 includes one or more processors 156, such as a digital processor, a microprocessor, an electronic processor, an optical processor, a multi-processor, a distribution of processors including peer-to-peer or cooperatively operating processors, client-server arrangement of processors, and the like. The console 150 includes computer readable storage medium (“memory”) 158, which excludes transitory medium.

The therapy control device 122 is suitably embodied by one or more configured processors, such as the processors 156 of the console 150. The configured processor 156 executes at least one computer readable instruction stored in computer readable storage medium, such as the memory 158 of the console 150, which excludes transitory medium and includes physical memory and/or other non-transitory medium to perform the disclosed temperature mapping, strain mapping, anatomical imaging generation, treatment failure mode detection and therapy device control. The configured processor may also execute one or more computer readable instructions carried by a carrier wave, a signal or other transitory medium. The configured processor can comprise a computing device 164, such as a workstation, laptop, tablet, smart phone, body worn computing device, server, and the like. The lines between components represented in the exemplary diagram represent communications paths, which can be wired or wireless.

In some embodiments, the therapy control device 112 is communicatively connected to a therapy planning system (not shown), which receives notice of the detected treatment failure modes and/or delivery of the ultrasound energy. The received notice and/or delivery can be used as an aid in re-planning treatments. For example, the therapy control device can send the notice of the treatment failures and/or data of the delivery of the ultrasound energy, such as actual delivery of duration and intensity before treatment failure, and the therapy planning system can adjust a plan or re-plan treatments for patient based on the treatment failures, and/or data.

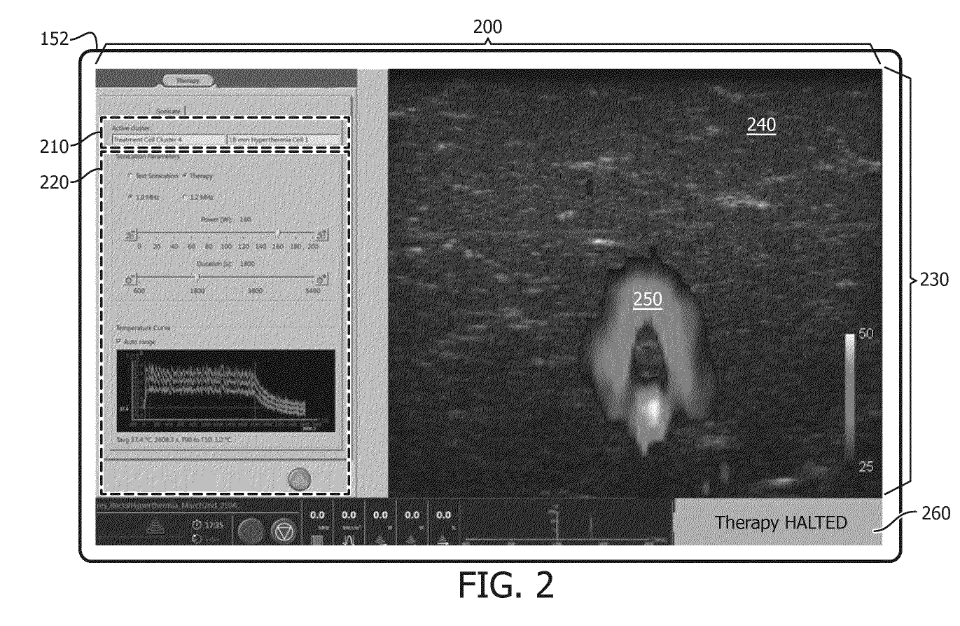

With reference to FIG. 2, an example system graphical user interface 200 of the ultrasound hyperthermia treatment system with detection of treatment failure modes is illustrated. The user interface 200 includes identification information 210, such as the target tissue identification, patient identification, and the like. The user interface 200 includes parameter settings and controls 220 for sonication or delivery of the ultrasound energy to the target tissue 114, such as power settings, predetermined timer settings, temperature ranges, start/stop treatment control, and the like. The user interface 200 includes the one or more input devices 154, which allow the healthcare practitioner to interact with the system 100.

The user interface 200 includes an image display area 230. The image display area 230 displays the anatomical image 240 with an overlay of a temperature map 250. The display area 230 can display combinations of the anatomical image 240, visualizations of the temperature map 140, visualizations of the strain map 142, and/or the cavitation map. For example, the combination of the anatomical image 240 with a visualization of the temperature map 140 includes temperatures different from normal body temperatures contrasted by color, texture, symbol, and the like. In some embodiments, the image display area 230 can include multiple different views, each in a sub-window. For example, each view represents a different view direction of a three dimensional image, which can include a planning image. Real-time therapy images can overlay or superimpose in each view direction with the temperature map 140 and/or strain map 142 according to a view plane. In another example, each view corresponds to a different spatial location and/or a different time, such as the current therapy time, a start of the therapy time, a first spatial location reaching temperatures of mild hyperthermia treatment, a maximum volume reaching temperatures of mild hyperthermia treatment, and the like.

The user interface 200 includes a message area 260. The message area 260 can include treatment failure mode warning messages and/or treatment failure mode halt messages. A treatment failure mode halt message is displayed simultaneously with the therapy control device 122 turning off or powering down the HIFU transducer 116. The imaging system 130 can continue to generate image data, which is displayed in the display area 230.

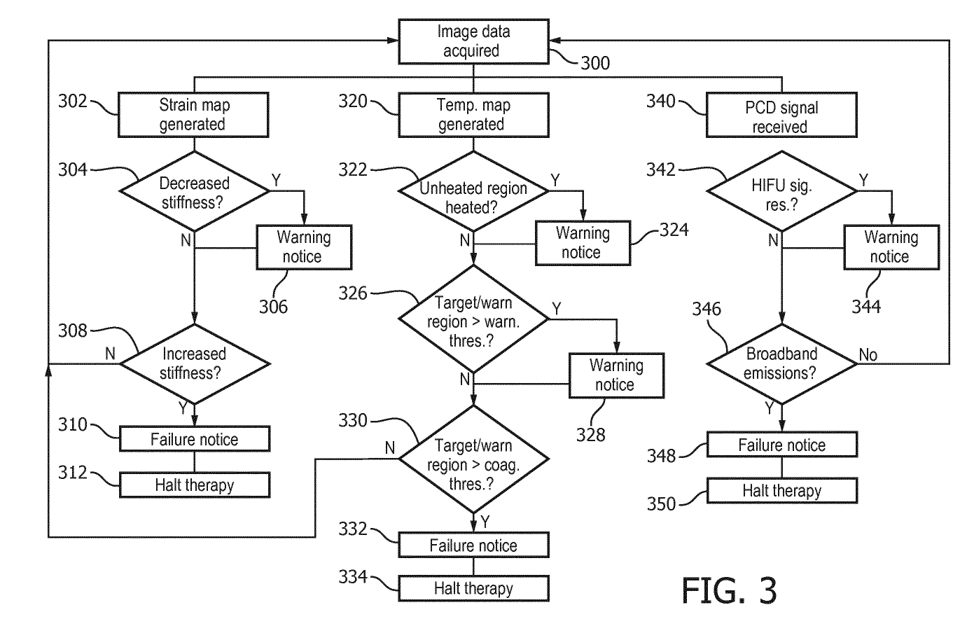

With reference to FIG. 3, an embodiment of a method for detecting treatment failure mode for mild hyperthermia is illustrated in a flowchart.

At 300, image data of the patient 112 is acquired. The image data can be

At 302, the strain map 142 is received.

At 304, the strain map 142 is compared with a prior strain map 142 for decreasing tissue stiffness values in the target region 114 and the warning regions 118. In one embodiment the strain map 142 is evaluated for decreasing tissue stiffness according to a normalized threshold for the tissues of the patient in the target region 114 and the warning regions 118.

In response to decreasing tissue stiffness (or increasing elasticity), a warning notification is provided through the user interface 200 at 306, such as a notice in the message area 260 that thermal damage may occur, a contrast of the spatial regions showing decreasing stiffness in the image display area 230, and/or the like. In some instances, tissues about to be thermally damage exhibit a decrease in tissue stiffness.

At 308, the strain map 142 is compared with the prior strain map 142 for increasing tissue stiffness values in the target region 114 and the warning regions 118. In one embodiment the strain map 142 is evaluated for increasing tissue stiffness according to a normalized threshold for the tissues of the patient in the target region 114 and the warning regions 118.

In response to increasing tissue stiffness (or decreasing elasticity), a failure notification is provided through the user interface 200 at 310, such as a notice in the message area 260 that thermal damage has occurred, a contrast of the spatial regions showing decreased stiffness or damaged regions in the image display area 230, and/or the like. The therapy control device 122 halts the therapy at 312. In some instances, tissues thermally damaged exhibit an increase in tissue stiffness. In response to no increase in tissue stiffness, processing continues with acquiring new image data at 300.

At 320, the temperature map 140 is received.

At 322, untreated regions 120 according to the temperature map 140 are compared to a predetermined unheated region detection threshold value. For example a value greater than 38° C. or other value above normal body temperature can be used.

In response to one or more voxels in the untreated regions 120 greater than the predetermined unheated region detection threshold value, a warning notification is provided through the user interface 200 at 324, such as a notice in the message area 260 that unintended heating has occurred, a contrast of the spatial regions showing unintended heating in the image display area 230, and/or the like.

At 326, the target region 114 and/or the warning region 118 according to the temperature map 140 are compared to a predetermined temperature warning threshold value. For example, a predetermined temperature warning threshold value is greater than 44° C., 46° C. or other value above a range of 40-43° C., 40-45° C., etc.

In response to one or more voxel temperature values in the target region 114 and/or the warning region 118 greater than the predetermined temperature warning threshold value, a warning notification is provided through the user interface 200 at 328, such as a notice in the message area 260 that thermal damage may occur, a contrast of the spatial regions showing temperatures above the predetermined temperature warning threshold value in the image display area 230, and/or the like.

At 330, the target region 114 and/or the warning region 118 according to the temperature map 140 are compared to a predetermined coagulation threshold value, such as 53° C.

In response to temperatures in one or more voxels in the target region 114 and/or the warning region 118 greater than the predetermined coagulation threshold value, a failure notification is provided through the user interface 200 at 332, such as a failure notice in the message area 260 that thermal damage has occurred, a contrast of the spatial regions showing damaged regions in the image display area 230, and/or the like. In response to the temperatures in the target region 114 and the warning region 118 not greater than the predetermined coagulation threshold value, processing continues or repeats at 300. The therapy control device 122 halts therapy at 334. In some instances, tissues thermally damaged exhibit high temperatures.

At 340, the PCD signal is received.

At 342, a frequency of the PCD signal is compared to a frequency of the HIFU transducer 116.

In response to PCD signal frequency being a harmonic of the frequency of the HIFU transducer 116, such as one half, a warning notification is provided through the user interface 200 at 344, such as a notice in the message area 260 that inertial cavitation may occur, a contrast of the spatial regions (according to the cavitation map) showing potential inertial cavitation in the image display area 230, and/or the like. In some instances, bubble formation occurs with the inertial cavitation, and the bubbles oscillate in a frequency related to the HIFU transducer 116, which are detected by the PCD device 146, such as a harmonic frequency.

At 346, the PCD signal is evaluated for broadband emissions. In response to the PCD signal including broadband emissions, a failure notification is provided through the user interface 200 at 348, such as a notice in the message area 260 that inertial cavitation has occurred, a contrast of the spatial regions (according to the cavitation map) showing inertial cavitation in the image display area 230, and/or the like. In response to no broadband emissions detected according to the PCD signal, processing continues or repeats at 300.

The therapy control device 122 halts therapy at 348. In some instances, bubble implosions occur with the inertial cavitation, which are detected by the PCD device 146.

The processing between 302-312, 320-334 and 340-350 can be concurrent or in parallel. For example, the failure determined according to increased stiffness measured by the strain map 142 at 308, the failure determined according to the target region 114 and/or the warning region 118 greater than the predetermined coagulation threshold at 330, or the failure determined according to the detected broadband emissions at 346 can be perform concurrently.

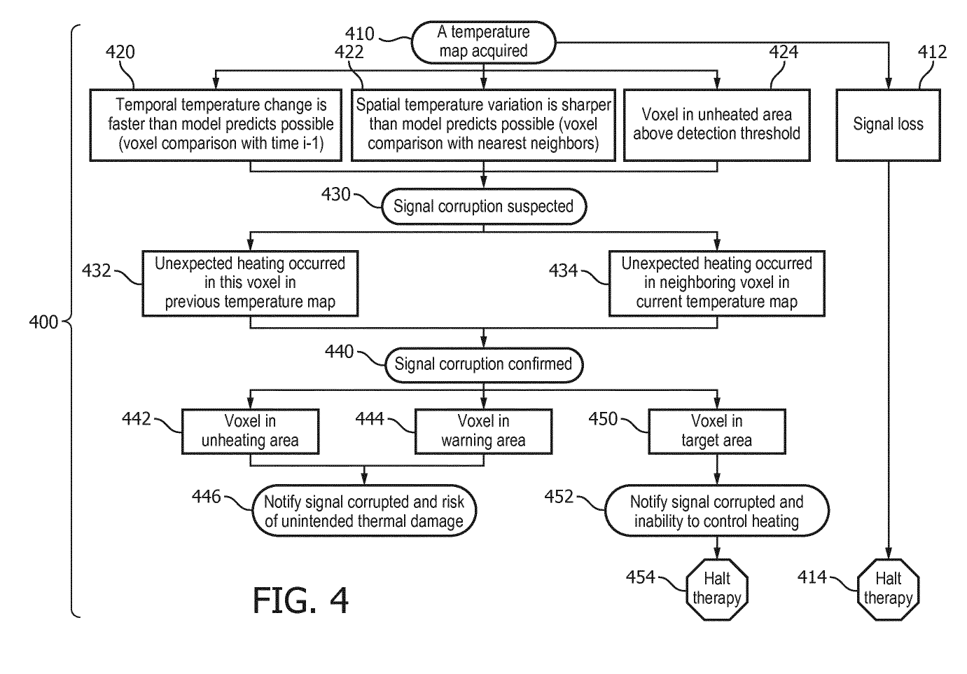

With reference to FIG. 4, an embodiment of a method for detecting a failure mode for mild hyperthermia is flowcharted. A fourth flowchart 400 detects a treatment failure mode according to image monitoring signal loss or corruption from the generated imaging data of the imaging system 130.

At 410, the temperature map 140 is received from the imaging system 130. The imaging system can include either of the MR imaging system 134 or the US imaging system 132.

At 412, the therapy control device 122 evaluates the signal from the imaging system 130 for signal loss. For example, no temperature map 140 is available. If no temperature map 140 is available, then at 414 therapy is halted. The evaluation can include repeated attempts or tries at receiving the temperature map 140. The evaluation can include a predetermined time after which in the event of no temperature map 140 received that therapy is halted.

At 420, the received temperature map 140 is compared with a prior temperature map, and temperature differences are evaluated by voxel(s) for changes or rates of change greater than predicted by a model. For example, a first model, such as from a linear regression, based on the previous temperatures and the energy of the HIFU transducer 116 can be used to predict an expected temperature change. Changes greater than a threshold margin, such as one or more standard deviations or a predetermined fixed range above the expected changes can be determined as possible signal corruption.

At 422, the received temperature map 140 is evaluated for spatial variations greater than a predetermined threshold value or a second model prediction. The spatial variations include voxel temperature comparisons with nearest neighbors. For example, a voxel is compared with a nearest neighbor for a threshold temperature difference of 5° C. The second model can include a distance measurement for the predetermined threshold value, such as a temperature difference of 5° C. within a neighboring distance of 20 voxels. The second model can include factors or operating aspects of the HIFU transducer 116, such as the focal region, power, frequency, and the like. If a temperature variation for a voxel location is determined to be greater that a predetermined threshold value or greater than the second model prediction including a margin of error, such as one or more standard deviations, then possible signal corruption is determined.

At 424, temperatures of voxels in the temperature map 140 corresponding to the unheated region 120 are evaluated for temperatures above a predetermined unheated detection threshold. If temperatures above the predetermined detection unheated threshold are detected for one or more voxels, then possible signal corruption is determined.

Acts 420, 422 and 424 can be performed in series, concurrently or in parallel. At 430, if no possible signal corruption is determined for all of the acts, then processing continues with the receiving of a next temperature map. In some instances, the possible signal corruption occurs due to patient motion, poor contact between the imaging system and the patient, or data signal corruption.

If possible signal corruption is determined for any one of the acts 420, 422 or 424, then processing continues at 432, where the voxel at spatial location (x, y, z) determined with possible signal corruption is analyzed with respect to the previous temperature map at spatial location (x, y, z) for unexpected heating according to any one of 420, 422, or 424.

If possible, signal corruption is determined for any of the acts 420, 422 or 424, then processing continues concurrently or in parallel at 434, where neighboring voxels to the determined voxel at spatial location (x, y, z) is analyzed with respect to the current temperature map for unexpected heating according to any one of 420, 422, or 424.

At 440, if the analysis of the corresponding voxel in a prior temperature map or the analysis of neighboring voxels in a current temperature map indicate unexpected heating has occurred, then signal corruption is confirmed. If the analysis does not indicate unexpected heating has occurred, then processing repeats or continues at 410.

At 442, if the spatial location of the voxels with unexpected heating are located in the unheated region 120, then a notification is provided through the user interface 200 at 446, such as a notice in the message area 260 that the imaging signal is corrupted for unheated regions and a risk exists of unintended thermal damage, a contrast of the spatial regions showing signal corruption in the unheated regions 120 of the image display area 230, and/or the like.

At 444, if the spatial location of the voxels with unexpected heating are located in the warning region 118, then a notification is provided through the user interface 200 at 446, such as a notice in the message area 260 that the imaging signal is corrupted for warnings regions and a risk exists of unintended thermal damage, a contrast of the spatial regions showing signal corruption in the warning regions 120 of the image display area 230, and/or the like.

At 450, if the spatial location of the voxels with unexpected heating are located in the target region 114, then a notification is provided through the user interface 200 at 452, such as a notice in the message area 260 that the imaging signal is corrupted for the target region and an inability to control heating, a contrast of the spatial regions showing signal corruption in the target region 120 of the image display area 230, and/or the like. At 454, the therapy is halted for any voxels in the target region 114 confirmed with signal corruption.

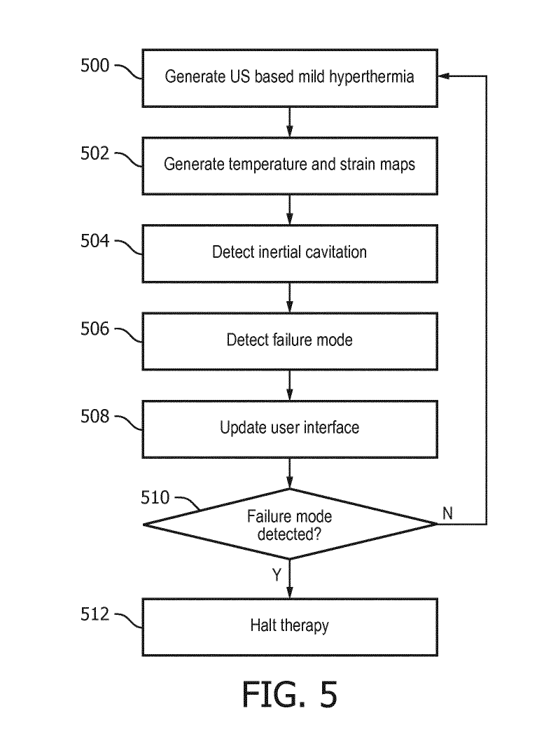

With reference to FIG. 5, an embodiment of a method of ultrasound mild hyperthermia treatments with detected treatment failure modes is flowcharted. At 500 US based mild hyperthermia is generated in the localized tissues 110 of the patient 112 by the

HIFU transducer 116.

At 502 the temperature map 140 and the strain map 142 are generated by the imaging system 130 of the localized tissues 110. The temperature map 140 and the strain map 142 can be generated by either of the MR imaging system 134 and/or the US imaging system 132. Anatomical images of the localized tissues 110 can be generated by the imaging system 130.

Inertial cavitation is detected in the localized tissues 110, and the PCD 146 generates a signal indicated of detected inertial cavitation at 504. The detected inertial cavitation can include harmonic frequencies of the HIFU transducer 116 indicative of bubble formation, such as half the frequency of the HIFU transducer 116. The detected inertial cavitation can include broadband emissions indicated of implosions.

At 506 one or more treatment failure modes are detected. The treatment failure modes can be detected concurrently or in parallel. The detected treatment failure modes can include increased tissue stiffness in the target region 114 and/or warning region 118 according to the strain map 142 as further described in FIG. 3. The detected treatment failure modes can include heating of the target region 114 and/or warning region 118 according to the temperature map 140 as further described in FIG. 3. The detected treatment failure modes can include inertial cavitation according to the signal generated by the PCD 146 as further described in FIG. 3. The detected treatment failure modes can include signal corruption and/or signal loss according to the temperature maps 140 as further described in FIG. 4. The detected treatment failure modes can include detected treatment failure mode warnings, which are further described in FIGS. 3-4.

At 508 the user interface 200 is updated. The update includes updating the image display area 230, such as the anatomical image 240, an overlay or contrast of the temperature map 140 and/or strain map 142. The update can include updating the message display area 260 according to a detected failure mode or a failure mode warning message.

At 510, if one or more treatment failure modes are detected, then therapy is halted at 512. If one or more treatment failure modes are not detected, then processing can continue or repeat at 500.

In some embodiments, the therapy planning can send notice of the detected treatment failure modes and/or data regarding delivery of the ultrasound energy and a therapy planning system, and used the received notice and/or data about delivery to aid in re-planning

The above may be implemented by way of computer readable instructions, encoded or embedded on computer readable storage medium, which, when executed by a computer processor(s), cause the processor(s) to carry out the described acts. Additionally or alternatively, at least one of the computer readable instructions is carried by a signal, carrier wave or other transitory medium.

The invention has been described with reference to the preferred embodiments. Modifications and alterations may occur to others upon reading and understanding the preceding detailed description. It is intended that the invention be constructed as including all such modifications and alterations insofar as they come within the scope of the appended claims or the equivalents thereof.

1. A system, comprising:
an imaging system configured to generate temperature maps and strain maps of localized tissues of a patient;
a passive cavitation detector configured to detect the inertial cavitation within the localized tissues of the patient and to generate a signal indicative of the detected inertial cavitation; and
a therapy control device comprising one or more computer processors configured to detect at least one treatment failure mode of generated mild hyperthermia in the localized tissues of the patient according to the temperature maps, the strain maps, and the signal indicative of detected inertial cavitation.

2. The system according to claim 1, wherein the detected at least one treatment failure mode includes at least one selected from a group comprising of:
an inertial cavitation according to the signal indicated of the detected inertial cavitation; and
a signal corruption according to the temperature maps.

3. The system according to claim 1, wherein the imaging system includes an ultrasound imaging system configured to generate the temperature maps, the strain maps, and an anatomical image of the localized tissues of the patient.

4. The system according to claim 1, further comprising:
a high intensity focused ultrasound (HIFU) transducer configured to generate and maintain the mild hyperthermia with high intensity focused ultrasound in the localized tissues of the patient, wherein generated mild hyperthermia comprises heating of the localized tissues of the patient to 40-45° C. for 10-60 minutes and excludes ablative treatment.

5. The system according to claim 1, wherein the therapy control device is further configured to, in response to the detected at least one treatment failure mode, halt the generated mild hyperthermia and display a message on a display device.

6. The system according to claim 4, wherein the HIFU is configured to focus the high intensity ultrasound at a target region within the localized tissues of the patient, and the high intensity ultrasound traverses a warning region within the localized tissues of the patient, wherein the localized tissues of the patient include unheated regions adjacent the target region and the warning region.

7. The system according to claim 6, wherein the therapy control device is configured to halt the high intensity focused ultrasound in response to at least one selected from a group comprised of:
an increased stiffness values in the strain map corresponding to one or more voxels in the target region above a predetermined threshold for the patient;
an increased stiffness values in the strain map corresponding to one or more voxels in the warning region above a predetermined threshold for the patient;
an increased stiffness change in values in a current strain map from a previous strain map corresponding one or more voxels in the target region above a predetermined threshold; and
an increased stiffness change in values in a current strain map from a previous strain map corresponding one or more voxels in the warning region above a predetermined threshold.

8. The system according to claim 6, wherein the therapy control device is configured to halt the high intensity focused ultrasound in response to at least one selected from a group comprised of:
temperatures in one or more voxels corresponding one or more voxels in the target region according to the temperature maps which exceed a predetermined coagulation threshold; and
temperatures in one or more voxels corresponding one or more voxels in the warning region according to the temperature maps which exceed a predetermined coagulation threshold.

9. The system according to claim 6, wherein the therapy control device is configured to halt the high intensity focused ultrasound in response to at least one selected from a group comprised of:
unexpected heating in one or more voxels corresponding one or more voxels in the target region according to the temperature maps; and
unexpected heating in one or more neighboring voxels corresponding one or more voxels in the target region according to the temperature maps.

10. The system according to claim 9, wherein unexpected heating includes at least one selected from a group comprised of:
a temperature change between at least one voxel of two temporally related temperature maps which exceeds a predicted value according to a first model; and
a temperature difference between the at least one voxel and at least one nearest neighboring voxel which exceeds a predicted value according to a second model.

11. A method, comprising:
generating temperature maps and strain maps of localized tissues of a patient;
detecting the inertial cavitation within the localized tissues of the patient which generates a signal indicative of the detected inertial cavitation; and
detecting at least one treatment failure mode of generated mild hyperthermia in the localized tissues of the patient according to the temperature maps, the strain maps, and the signal indicative of detected inertial cavitation.

12. The method according to claim 11, wherein detecting at least one treatment failure mode includes detecting at least one from a group comprising of:
an inertial cavitation according to the signal indicated of the detected inertial cavitation; and
a signal corruption according to the temperature maps.

13. The method according to claim 11, further including:
in response to the detected at least one treatment failure mode, halting the generated mild hyperthermia and displaying a message on a display device.

14. The method according to claim 11, further including:
generating and maintaining the mild hyperthermia with high intensity focused ultrasound in the localized tissues of the patient, wherein generated mild hyperthermia comprises heating of the localized tissues of the patient to 40-45° C. for 10-60 minutes and excludes ablative treatment;
and
wherein generating mild hyperthermia includes focusing the high intensity ultrasound at a target region within the localized tissues of the patient, and the high intensity ultrasound traverses a warning region within the localized tissues of the patient, wherein the localized tissues of the patient include unheated regions adjacent the target region and the warning region.

15. The method according to claim 13, wherein halting the high intensity focused ultrasound includes a response to at least one selected from a group comprised of:
an increased stiffness values in the strain map corresponding one or more voxels in the target region or in the warning region above a predetermined threshold for the patient; and
an increased stiffness change in values in a current strain map from a previous strain map corresponding one or more voxels in the target region or in the warning region above a predetermined threshold.

16. The method according to claim 13, wherein halting the high intensity focused ultrasound includes a response to at least one selected from a group comprised of:
temperatures in one or more voxels corresponding to one or more voxels in the target region according to the temperature maps exceeding a predetermined coagulation threshold; and
temperatures in one or more voxels corresponding to one or more voxels in the warning region according to the temperature maps exceeding a predetermined coagulation threshold.

17. The method according to claim 13, wherein halting the high intensity focused ultrasound includes a response to at least one selected from a group comprised of:
unexpected heating in one or more voxels corresponding one or more voxels in the target region according to the temperature maps; and
unexpected heating in one or more neighboring voxels corresponding one or more voxels in the target region according to the temperature maps.

18. The method according to claim 17, wherein unexpected heating includes at least one selected from a group comprised of:
a temperature change between at least one voxel of two temporally related temperature maps which exceeds a predicted value according to a first model; and
a temperature difference between the at least one voxel and at least one nearest neighboring voxel which exceeds a predicted value according to a second model.

19. The method according to claim 11, wherein the steps are performed by one or more configured computer processors.

20. A system, comprising:
a high intensity focused ultrasound (HIFU) transducer configured to generate mild hyperthermia with high intensity focused ultrasound in localized tissues of a patient;
an imaging system configured to generate temperature maps and strain maps of the localized tissues of the patient;
a passive cavitation detector configured to detect inertial cavitation within the localized tissues of the patient and to generate a signal indicative of the detected inertial cavitation; and
a therapy control device comprising one or more computer processors configured to detect at least one treatment failure mode of the generated of mild hyperthermia in the localized tissues of the patient according to the temperature maps, the strain maps, and the signal indicative of the detected inertial cavitation, wherein the therapy control device is further configured in response to the at least one detected treatment failure mode, halt the generated mild hyperthermia by the HIFU transducer.
